(19) United States
(12) Patent Application Publication
(10) Pub. No.: US 2026/0026869 A1
(43) Pub. Date: Jan. 29, 2026

(54) TREATMENT TOOL

(71) Applicant: OLYMPUS MEDICAL SYSTEMS CORP., Tokyo (JP)

(72) Inventors: Tsubasa NIIYAMA, Tokyo (JP); Marina Asahina, Sagamihara-shi (JP); Keisuke Nagao, Tokyo (JP); Kaito Sato, Tokyo (JP)

(21) Appl. No.: 18/990,441

(22) Filed: Dec. 20, 2024

Related U.S. Application Data
(63) Continuation of application No. PCT/JP2024/026879, filed on Jul. 26, 2024.

(51) Int. Cl. A61B 18/14; A61B 18/00

(52) U.S. Cl. CPC A61B 18/1445; A61B 2018/00077; A61B 2018/00083; A61B 2018/00178; A61B 2018/00184; A61B 2018/00994

(57) ABSTRACT

A treatment tool includes: a vibration transmission portion having a distal end provided with a treatment portion that functions as a first electrode supplying a high frequency current to a biological tissue; a jaw configured to be opened and closed with respect to the treatment portion, the jaw functioning as a second electrode supplying the high frequency current to the biological tissue; a pipe through which the vibration transmission portion is inserted with the treatment portion protruding from a distal end of the pipe, the pipe including an electrically conductive path to the second electrode; and a support provided on an inner surface of an end portion of the pipe on a distal end side, the support being configured to cover an outer surface of the end portion of the pipe on the distal end side and support the vibration transmission portion.

### CROSS-REFERENCE TO RELATED APPLICATION

This application is a continuation of International Application No. PCT/JP2024/026879, filed on July 26, 2024, the entire contents of which are incorporated herein by reference.

### BACKGROUND

### Technical Field

The present disclosure relates to a treatment tool.

### Related Art

In the related art, there is a known treatment tool that performs treatment on a region targeted for treatment of biological tissue (hereinafter, referred to as a treatment target) by supplying ultrasound vibration and a high frequency current as treatment energy to the treatment target (for example, see Japanese Laid-open Patent Publication No. 2009-240773).

The treatment tool described in Japanese Laid-open Patent Publication No. 2009-240773 includes a vibration transmission portion, a jaw, and a pipe that are described below.

The vibration transmission portion includes a treatment portion that is provided at the distal end of the vibration transmission portion, that functions as a first electrode that supplies a high frequency current to the treatment target, and that supplies ultrasound vibration to the treatment target.

The jaw is opened and closed with respect to a treatment portion, and functions as a second electrode that supplies a high frequency current to the treatment target.

The pipe includes an electrically conductive path to the second electrode and through which the vibration transmission portion is inserted.

### SUMMARY

In some embodiments, a treatment tool includes: a vibration transmission portion having a distal end provided with a treatment portion that functions as a first electrode supplying a high frequency current to a biological tissue, the treatment portion being configured to supply ultrasound vibration to the biological tissue; a jaw configured to be opened and closed with respect to the treatment portion, the jaw functioning as a second electrode supplying the high frequency current to the biological tissue; a pipe through which the vibration transmission portion is inserted with the treatment portion protruding from a distal end of the pipe, the pipe including an electrically conductive path to the second electrode; and a support provided on an inner surface of an end portion of the pipe on a distal end side, the support being configured to cover an outer surface of the end portion of the pipe on the distal end side and support the vibration transmission portion, the support being made of an electrical insulating material such that a portion between the end portion of the pipe on the distal end side and the treatment portion is electrically insulated to prevent a short circuit from occurring by way of another electrically conductive portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram illustrating a configuration of a distal end portion of a treatment tool;

FIG. 3 is a diagram illustrating a first modification of the embodiment;

FIG. 4 is a diagram illustrating the first modification of the embodiment;

FIG. 5 is a diagram illustrating a second modification of the embodiment;

FIG. 6 is a diagram illustrating a third modification of the embodiment;

FIG. 7 is a diagram illustrating a fourth modification of the embodiment;

FIG. 8 is a diagram illustrating a sixth modification of the embodiment;

FIG. 9 is a diagram illustrating the sixth modification of the embodiment;

FIG. 10 is a diagram illustrating a seventh modification of the embodiment; and

FIG. 11 is a diagram illustrating an eighth modification of the embodiment.

### DETAILED DESCRIPTION

Modes (hereinafter, embodiments) for carrying out the present disclosure will be described below with reference to the drawings. Furthermore, the present disclosure is not limited to the embodiments described below. In addition, in description of the drawings, components that are identical to those in drawings are assigned the same reference numerals.

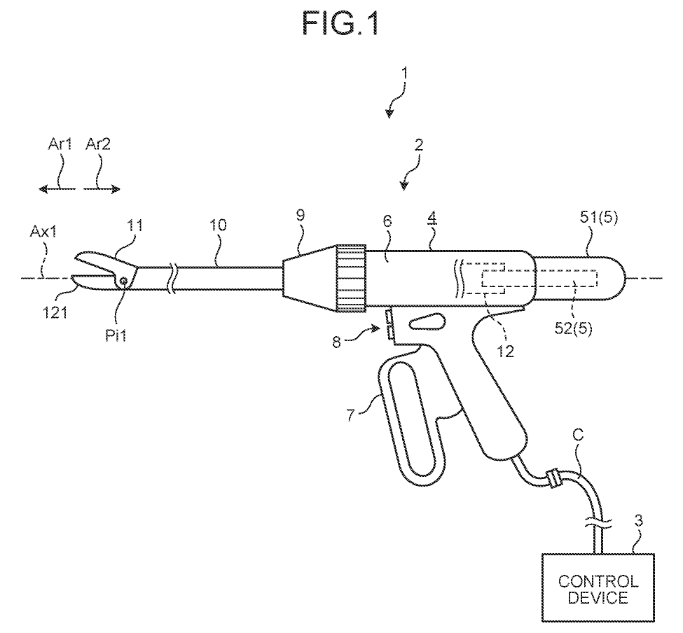

Regarding schematic configuration of treatment system FIG. 1 is a diagram illustrating a treatment system 1 according to an embodiment. The treatment system 1 performs treatment on a region targeted for treatment of biological tissue (hereinafter, referred to as a treatment target) by applying treatment energy to the treatment target. The treatment energy described in the present embodiment is ultrasound energy and high frequency energy. Furthermore, the treatment that is able to be performed by the treatment system 1 according to the present embodiment is treatment including coagulation (sealing) of the treatment target, incision of the treatment target, or the like. Moreover, both coagulation and incision may be performed at the same time. The treatment system 1 includes, as illustrated in FIG. 1, a treatment tool 2 and a control device 3.

Regarding configuration of treatment tool Furthermore, in the following description, one of the sides along a central axis Ax1 (FIG. 1) of an outer pipe 10 is referred to as a distal end side Ar1, whereas the other of the sides is referred to as a proximal end side Ar2. In addition, a "width direction" described below is a direction orthogonal to both of the central axis Ax1 and an opening/closing direction (in FIG. 1, in the upward and downward directions) of a jaw 11 with respect to a treatment portion 121, and indicates the direction orthogonal to the plane of the drawing in FIG. 1.

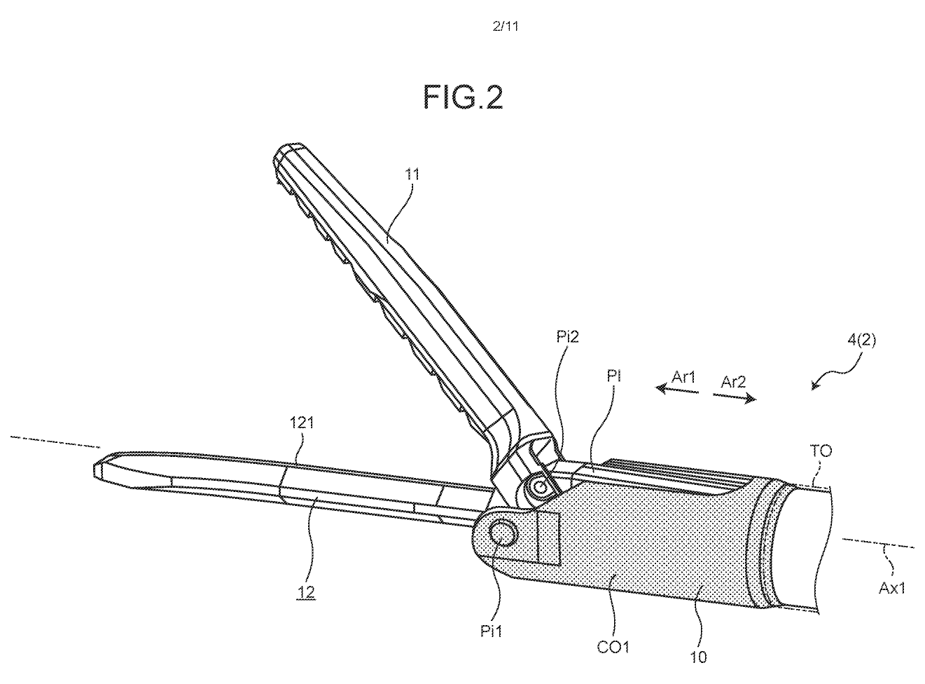

FIG. 2 is a diagram illustrating a configuration of a distal end portion of the treatment tool 2. Specifically, FIG. 2 is a perspective view indicating the distal end portion of the treatment tool 2. The treatment tool 2 performs treatment on the treatment target by applying ultrasound energy and high frequency energy to the treatment target. The treatment tool 2 includes, as illustrated in FIG. 1, a handpiece 4 and an ultrasound transducer 5.

The handpiece 4 includes, as illustrated in FIG. 1 and FIG. 2, a fixed handle 6 (FIG. 1), an operation handle 7 (FIG. 1), a switch 8 (FIG. 1), a rotatable knob 9 (FIG. 1), the outer pipe 10, the jaw 11, and a vibration transmission portion 12.

The fixed handle 6 is a portion that supports the entire of the treatment tool 2, and that is gripped by an operator (user), such as a technician.

The operation handle 7 is attached to the fixed handle 6 such that the operation handle 7 is able to move with respect to the fixed handle 6, and receives an opening/closing operation performed by the operator, such as the technician.

The switch 8 is provided such that the switch 8 is externally exposed to the outside from the fixed handle 6, and receives a treatment operation performed by the operator, such as the technician.

The rotatable knob 9 is formed to have a substantially cylindrical shape that is coaxial with the central axis Ax1, and is provided at the fixed handle 6 on the distal end side Ar1. Then, the rotatable knob 9 receives a rotation operation performed by the operator, such as the technician. As a result of the rotation operation, the rotatable knob 9 rotates about the central axis Ax1 relative to the fixed handle 6. Furthermore, as a result of the rotation of the rotatable knob 9, the outer pipe 10, the jaw 11, and the vibration transmission portion 12 also rotates about the central axis Ax1.

The outer pipe 10 corresponds to a pipe and a first pipe. The outer pipe 10 is a pipe that is formed in a cylindrical shape, that is made of an electrically conductive material, such as metal, and that forms an electrically conductive path. In the outer pipe 10, a first pin Pi1 (FIG. 1 and FIG. 2) that has a columnar shape, that extends in the direction orthogonal to the plane of the drawing in FIG. 1, that is engaged with the jaw 11 and pivotally and rotatably supports the jaw 11 is fixed at the end portion located on the distal end side Ar1. In the present embodiment, the first pin Pi1 is made of an electrically conductive material, such as metal.

Then, the outer surface of the outer pipe 10 is covered by an outer tube TO (FIG. 2) that is made of the electrical insulating material. The outer tube TO corresponds to a tube. Moreover, in FIG. 2, the outer tube TO is indicated by an alternate long and short dash line. Furthermore, an inner pipe PI (FIG. 2) that is formed in a tubular shape and that moves forward and backward along the longitudinal direction of the outer pipe 10 in accordance with an opening/closing operation on the operation handle 7 performed by the operator, such as the technician, is inserted in the interior of the outer pipe 10. The inner pipe PI is made of an electrically conductive material, such as metal. Then, the inner pipe PI corresponds to a second pipe. Furthermore, a second pin Pi2 (FIG. 2) that has a columnar shape extending in the direction orthogonal to the plane of the drawing in FIG. 1, and that is engaged with the jaw 11 is fixed to the end portion of the inner pipe PI located on the distal end side Ar1. In the present embodiment, the second pin Pi2 is made of an electrically conductive material, such as metal, and is arranged on the upper side of the first pin Pi1 in FIG. 2 (on the side in which the jaw 11 is arranged with respect to the treatment portion 121).

The jaw 11 is made of an electrically conductive material, such as metal, and is pivotally supported with respect to the outer pipe 10 by the first pin Pi1. In other words, the jaw 11 is electrically connected to the outer pipe 10 via the first pin Pi1. Furthermore, the jaw 11 is connected to the inner pipe PI by the second pin Pi2. Then, the jaw 11 pivots about the first pin Pi1 with respect to the outer pipe 10 in conjunction with the forward and backward movements of the inner pipe PI in accordance with the opening/closing operation performed on the operation handle 7 by the operator, such as the technician. As a result of this, the jaw 11 is opened and closed with respect to the treatment portion 121 corresponding to the end portion of the vibration transmission portion 12 located on the distal end side Ar1, and enables the treatment target to be gripped between the treatment portion 121 and the jaw 11. Moreover, the treatment tool 2 may be configured to have a push close type, or may be configured to have a pull close type.

The configuration of the push close type is as follows. The jaw 11 pivots about the first pin Pi1 in a direction closer to the treatment portion 121 in conjunction with the movement of the jaw 11 to the inner pipe PI that is located on the distal end side Ar1. In other words, the jaw 11 is closed with respect to the treatment portion 121. Furthermore, the jaw 11 pivots about the first pin Pi1 in a direction away from the treatment portion 121 in conjunction with the movement of the jaw 11 to the inner pipe PI that is located on the proximal end side Ar2. In other words, the jaw 11 is opened with respect to the treatment portion 121.

The configuration of the pull close type is as follows. The jaw 11 pivots about the first pin Pi1 in a direction closer to the treatment portion 121 in conjunction with the movement of the jaw 11 to the inner pipe PI that is located on the proximal end side Ar2. In other words, the jaw 11 is closed with respect to the treatment portion 121. Furthermore, the jaw 11 pivots about the first pin Pi1 in a direction away from the treatment portion 121 in conjunction with the movement of the jaw 11 to the inner pipe PI that is located on the distal end side Ar1. In other words, the jaw 11 is opened with respect to the treatment portion 121.

The vibration transmission portion 12 is made of an electrically conductive material, and has a long shape that extends along the central axis Ax1. Furthermore, the vibration transmission portion 12 is inserted in the interior of the inner pipe PI (the outer pipe 10) in a state in which the treatment portion 121 externally protrudes from the inner pipe PI to the outside. At this time, as illustrated in FIG. 1, the end portion of the vibration transmission portion 12 located on the proximal end side Ar2 is mechanically connected to an ultrasound transducer 52 that constitutes the ultrasound transducer 5. Then, the vibration transmission portion 12 transmits the ultrasound vibration that has been generated by the ultrasound transducer 5 from the end portion of the vibration transmission portion 12 located on the proximal end side Ar2 to the treatment portion 121. The ultrasound vibration is longitudinal vibration that vibrates in a direction along the central axis Ax1. Furthermore, in the vibration transmission portion 12, the outer surface except for the treatment portion 121 is covered by an inner tube that is made of an electrically conductive material.

The ultrasound transducer 5 includes, as illustrated in FIG. 1, a transducer (TD) case 51 and the ultrasound transducer 52.

The TD case 51 supports the ultrasound transducer 52, and is connected to the fixed handle 6 so as to be capable of being attached to and removed from the fixed handle 6.

The ultrasound transducer 52 generates ultrasound vibration under the control of the control device 3. In the present embodiment, the ultrasound transducer 52 is constituted by a bolt-clamped Langevin-type transducer (BLT).

Regarding configuration of control device The control device 3 performs overall control of the operation of the treatment tool 2 by way of an electric cable C (FIG. 1).

Specifically, the control device 3 detects the treatment operation performed on the switch 8 by the operator, such as the technician, by way of the electric cable C. Then, when the control device 3 has detected the treatment operation, the control device 3 applies, by way of the electric cable C, treatment energy to the treatment target that is gripped between the jaw 11 and the treatment portion 121. In other words, the control device 3 performs treatment on the treatment target.

For example, when the control device 3 applies ultrasound energy to the treatment target, the control device 3 supplies driving power to the ultrasound transducer 52 by way of the electric cable C. As a result of this, the ultrasound transducer 52 generates longitudinal vibration (ultrasound vibration) that vibrates in the direction along the central axis Ax1. Furthermore, the treatment portion 121 vibrates at a desired amplitude by the longitudinal vibration. Then, the ultrasound vibration is applied from the treatment portion 121 to the treatment target that is gripped between the jaw 11 and the treatment portion 121. Accordingly, the ultrasound energy is applied from the treatment portion 121 to the treatment target.

Furthermore, for example, when high frequency energy is applied to the treatment target, the control device 3 supplies high frequency electrical power between the jaw 11 and the vibration transmission portion 12 by way of the electric cable C, or the like. Moreover, the electrically conductive path of the high frequency electrical power to the jaw 11 supplied by way of the electric cable C is a path passing through the electric cable C, the outer pipe 10, the first pin Pi1, and the jaw 11. Then, when the high frequency electrical power is supplied between the jaw 11 and the vibration transmission portion 12, a high frequency current is supplied to the treatment target that is gripped between the jaw 11 and the treatment portion 121. In other words, high frequency energy is applied to the treatment target. That is, the treatment portion 121 functions as the first electrode. Furthermore, the jaw 11 functions as the second electrode.

Regarding outer surface of end portion of outer pipe located on distal end side In the following, the outer surface of the end portion of the outer pipe 10 located on the distal end side Ar1 will be described with reference to FIG. 2. In the present embodiment, the outer surface of the outer pipe 10 is covered by the outer tube TO, but only the end portion of the outer pipe 10 located on the distal end side Ar1 is externally exposed to the outside. Furthermore, as illustrated in FIG. 2, a first coating layer CO1 that is made of an electrical insulating material is provided on the outer surface of the end portion of the outer pipe 10 that is located on the distal end side Ar1 and that is externally exposed to the outside. Moreover, in FIG. 2, the first coating layer CO1 is indicated by dots. An example of the electrical insulating material includes polyimide, polyetheretherketone (PEEK), or the like. In other words, in the present embodiment, a portion between the end portion of the outer pipe 10 located on the distal end side Ar1 and the treatment portion 121 is electrically insulated by the first coating layer CO1 in order to prevent a short circuit from occurring by way of another electrically conductive portion.

According to the above described present embodiment, the following effects are provided. In the treatment tool 2 according to the present embodiment, a portion between the end portion of the outer pipe 10 located on the distal end side Ar1 and the treatment portion 121 is electrically insulated in order to prevent a short circuit from occurring by way of another electrically conductive portion (for example, forceps, a staple, a wire, etc.). Therefore, with the treatment tool 2 according to the present embodiment, it is possible to prevent a spark from occurring as a result of coming into contact with the other electrically conductive portion.

In particular, in the present embodiment, a portion between the end portion of the outer pipe 10 located on the distal end side Ar1 and the treatment portion 121 is electrically insulated by the first coating layer CO1 in order to prevent a short circuit from occurring by way of the other electrically conductive portion. Consequently, it is possible to suitably implement an effect in that it is possible to prevent a spark from occurring as a result of coming into contact with the other electrically conductive portion with a simple configuration.

Other embodiments In the above, detailed descriptions of the preferred embodiments have been described; however, the disclosure is not limited to only the embodiments described above. In the above described embodiment, a first to an eighth modifications that will be described below may be used.

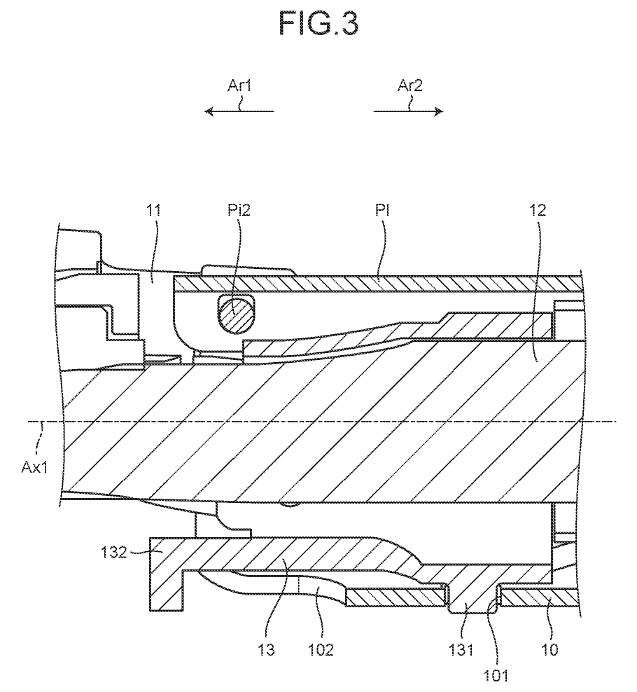

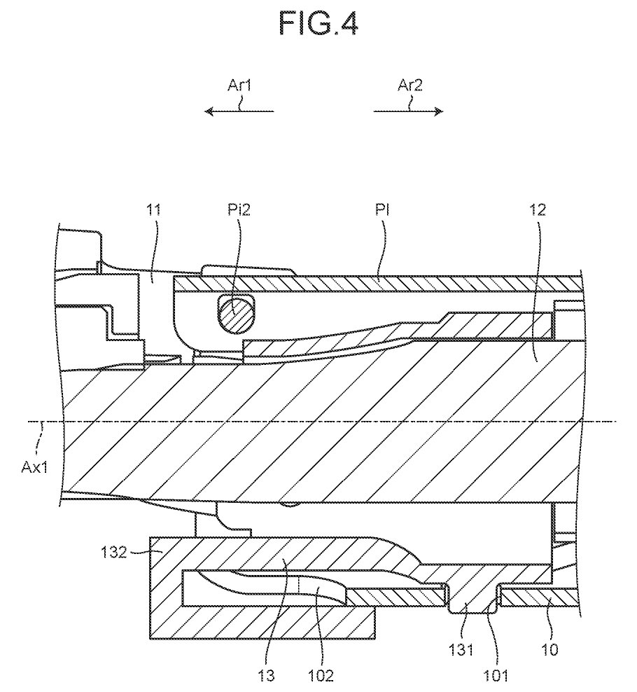

First Modification FIG. 3 and FIG. 4 are diagrams each illustrating a first modification of the embodiment. Specifically, FIG. 3 and FIG. 4 are cross-sectional view obtained by cutting treatment tool 2 according to the first modification by a plane that includes the central axis Ax1 at a position of the distal end portion of the outer pipe 10. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. As illustrated in FIG. 3 or FIG. 4, the portion between the end portion of the outer pipe 10 located on the distal end side Ar1 and the treatment portion 121 may be electrically insulated by a support 13 in order to prevent a short circuit from occurring by way of the other electrically conductive portion.

The support 13 is made of, for example, PEEK that is an electrical insulating material, and supports the vibration transmission portion 12. The support 13 has a cylindrical shape capable of allowing the vibration transmission portion 12 to be inserted, and is provided on the inner surface of the end portion of the outer pipe 10 located on the distal end side Ar1. By providing the support 13, even if the vibration transmission portion 12 is bent due to force applied from the jaw 11 when the jaw 11 is closed with respect to the treatment portion 121, it is possible to prevent the vibration transmission portion 12 from coming into contact with the inner surface of the outer pipe 10.

In the support 13, as illustrated in FIG. 3 or FIG. 4, a positioning protrusion 131 that protrudes from the outer surface outward in the radial direction is provided. The positioning protrusion 131 is a protrusion that is fitted to a positioning hole 101 provided in the outer pipe 10, and that performs positioning with respect to the outer pipe 10.

Here, as illustrated in FIG. 3 or FIG. 4, in the outer pipe 10, a notch portion 102 that is notched from the end surface of the distal end side Ar1 toward the proximal end side Ar2 is provided on the side opposite to the side in which the jaw 11 is located (the lower side in FIG. 1, FIG. 3, and FIG. 4). In addition, as illustrated in FIG. 3 or FIG. 4, in the support 13, a protrusion portion 132 is provided at the end portion of the distal end side Ar1 that is located on the side opposite to the side in which the jaw 11 is located.

In the example illustrated in FIG. 3, the protrusion portion 132 protrudes toward the distal end side Ar1, is bent toward the side opposite to the side in which the jaw 11 is located (the lower side in FIG. 3), and covers the end surface (the inner surface of the notch portion 102) of the outer pipe 10 located on the distal end side Ar1.

Furthermore, in the example illustrated in FIG. 4, the protrusion portion 132 protrudes toward the distal end side Ar1, is bent toward the side opposite to the side in which the jaw 11 is located (the lower side in FIG. 3), further extends toward the proximal end side Ar2 by being bent, and covers the outer surface of the outer pipe 10 at the end portion located on the distal end side Ar1.

Even in a case where the configuration of the above described first modification is used, effects similar to those described above in the embodiment are achieved.

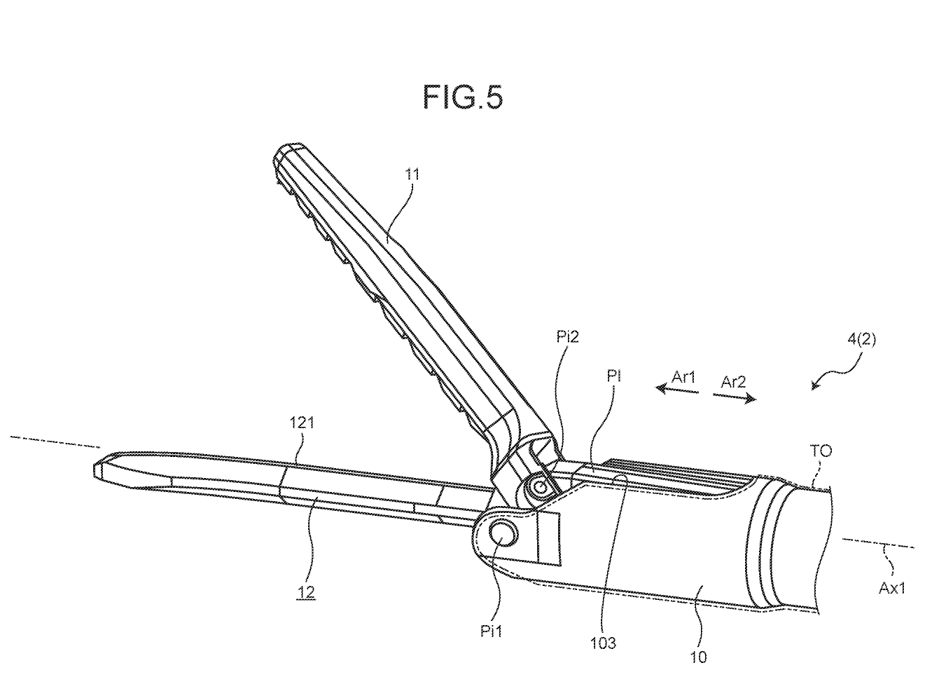

Second Modification FIG. 5 is a diagram illustrating a second modification of the embodiment. Specifically, FIG. 5 is a diagram corresponding to the diagram illustrated in FIG. 2. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. As described above in the second modification illustrated in FIG. 5, the portion between the end portion of the outer pipe 10 located on the distal end side Ar1 and the treatment portion 121 may be electrically insulated by the outer tube TO in order to prevent a short circuit from occurring by way of the other electrically conductive portion.

Specifically, as illustrated in FIG. 5, the outer tube TO covers the outer surface of the outer pipe 10 at the end portion located on the distal end side Ar1.

Here, as illustrated in FIG. 5, in the outer pipe 10, a notch portion 103 that is notched from the end surface of the distal end side Ar1 toward the proximal end side Ar2 is provided on the side in which the jaw 11 is located (the upper side in FIG. 5). Then, the outer tube TO covers the outer surface of the end portion of the outer pipe 10 located on the distal end side Ar1 except for the notch portion 103.

Even in a case where the configuration of the above described second modification is used, effects similar to those described above in the embodiment are achieved. Moreover, the outer tube TO may also cover the entire of the outer surface of the end portion of the outer pipe 10 located on the distal end side Ar1 (including the notch portion 103) as long as the outer tube TO prevents the jaw 11 from being opened and closed.

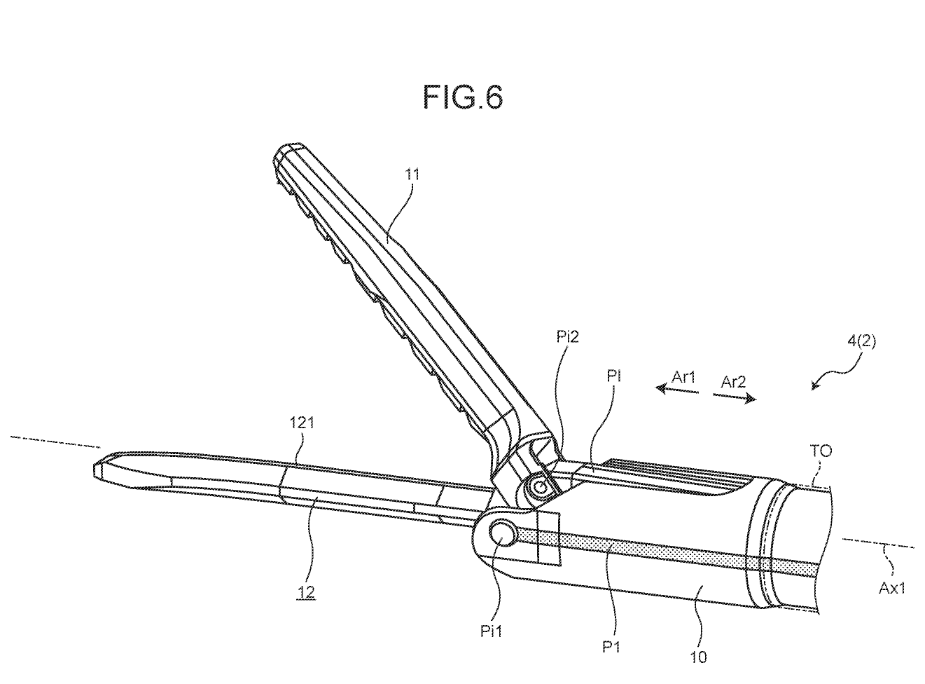

Third Modification FIG. 6 is a diagram illustrating a third modification of the embodiment. Specifically, FIG. 6 is a diagram corresponding to the diagram illustrated in FIG. 2. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. In the third modification, the outer pipe 10 is made of, for example, PEEK that is an electrical insulating material. As a result of this, the portion between the end portion of the outer pipe 10 located on the distal end side Ar1 and the treatment portion 121 is electrically insulated in order to prevent a short circuit from occurring by way of the other electrically conductive portion. In addition, as illustrated in FIG. 6, on the outer surface of the outer pipe 10, an electrically conductive path P1 for supplying high frequency electrical power to the jaw 11 is provided from the proximal end side Ar2 of the outer pipe 10 toward the distal end side Ar1. Moreover, in FIG. 6, the electrically conductive path P1 is indicated by dots.

In the third modification, the electrically conductive path P1 is formed by using a three-dimensional plating process. Specifically, the process is performed by irradiating the outer pipe 10 that has been made of the PEEK that is an electrical insulating material with a laser at a predetermined position. After that, the electrically conductive path P1 is formed at the irradiation position of the laser by performing an electroless plating process. Moreover, the thickness of the electrically conductive path P1 is about, for example, a few micrometers. Furthermore, the object generated by using this type of three-dimensional plating process is referred to as a molded interconnect device (MID) in which an electrode circuit is formed on the outer surface of a resin molded product having a three-dimensional shape. Moreover, the electrically conductive path for supplying high frequency electrical power to the jaw 11 by way of the electric cable C becomes a path passing through the electric cable C, the electrically conductive path P1, the first pin Pi1, and the jaw 11.

Moreover, the electrically conductive path P1 is not limited to the path that is formed by using the three-dimensional plating process described above, and a path that is formed in the outer pipe 10 by using another method may be used.

Even in a case where the configuration of the above described third modification is used, effects similar to those described above in the embodiment are achieved.

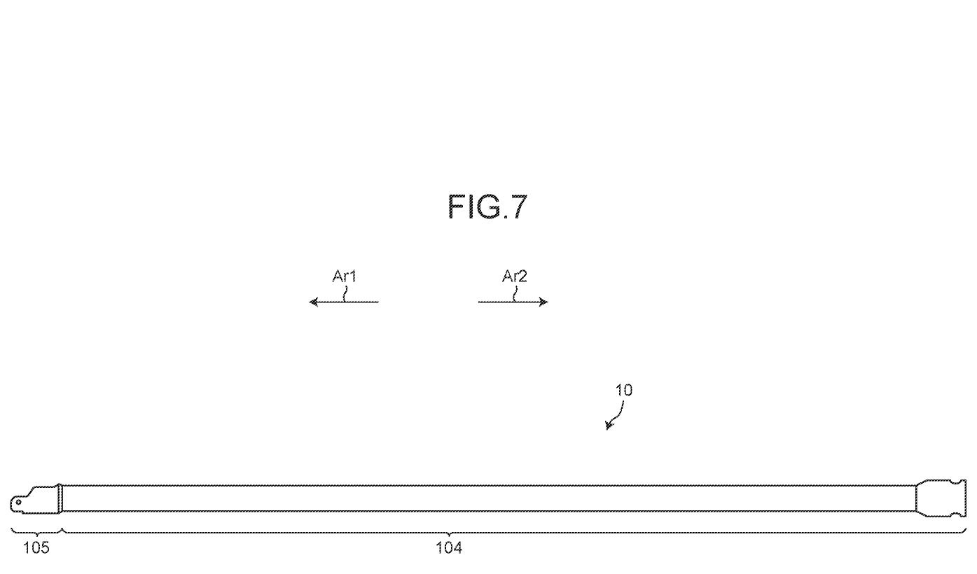

Fourth Modification FIG. 7 is a diagram illustrating a fourth modification according to the embodiment. Specifically, FIG. 7 is a diagram illustrating the outer pipe 10 according to the fourth modification. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. In the fourth modification, the outer pipe 10 is made of an electrically conductive material, such as metal, and includes a proximal end portion 104 that constitutes an electrically conductive path for supplying high frequency electrical power to the jaw 11, and a distal end portion 105 that is made of, for example, PEEK that is an electrical insulating material and that is provided at the end portion of the proximal end portion 104 located on the distal end side Ar1. As a result of this, a portion between the end portion (the distal end portion 105) of the outer pipe 10 located on the distal end side Ar1 and the treatment portion 121 is electrically insulated in order to prevent a short circuit from occurring by way of the other electrically conductive portion. Furthermore, the electrically conductive path for supplying high frequency electrical power to the jaw 11 by way of the electric cable C becomes an electrically conductive path (not illustrated) that is formed by using the three-dimensional plating process and that passes through the electric cable C, the proximal end portion 104, and the distal end portion 105, or becomes a path passing through the lead wire (not illustrated), the first pin Pi1, and the jaw 11.

Even in a case where the configuration of the above described fourth modification is used, effects similar to those described above in the embodiment are achieved.

Fifth Modification In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. In the fifth modification, the electrically conductive path for supplying the high frequency electrical power to the jaw 11 by way of the electric cable C corresponds to the path passing through the electric cable C, the inner pipe PI, the second pin Pi2, and the jaw 11. Furthermore, the outer pipe 10 is made of, for example, PEEK that is an electrical insulating material. As a result of this, the portion between the end portion of the outer pipe 10 (including the inner pipe PI) located on the distal end side Ar1 and the treatment portion 121 is electrically insulated in order to prevent a short circuit from occurring by way of the other electrically conductive portion.

Even in a case where the configuration of the above described fifth modification is used, effects similar to those described above in the embodiment are achieved. Moreover, the outer pipe 10 may be made of an electrically conductive material, such as metal, instead of an electrical insulating material. In this case, the second pin Pi2 is made of, for example, PEEK that is an electrical insulating material. In other words, the second pin Pi2 corresponds to an insertion portion. Furthermore, the insertion portion according to the disclosure is not limited to the second pin Pi2. In a case where the second pin Pi2 is made of an electrically conductive material, such as metal, a coating layer that is covered by, for example, polyimide or PEEK, that is an electrical insulating material may be used for the second pin Pi2.

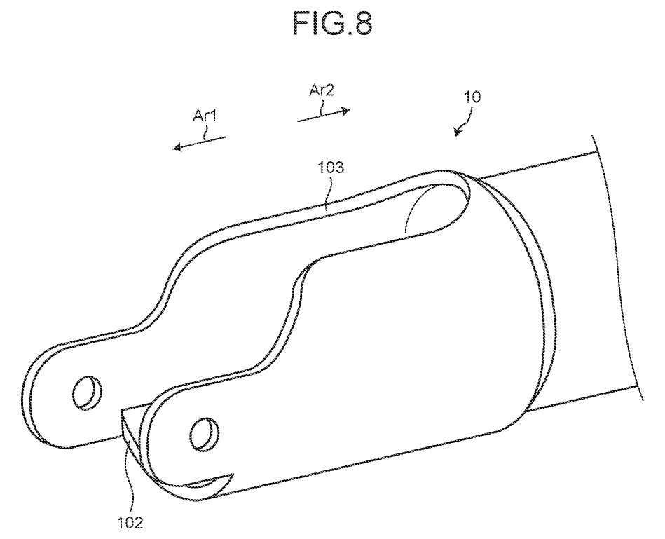

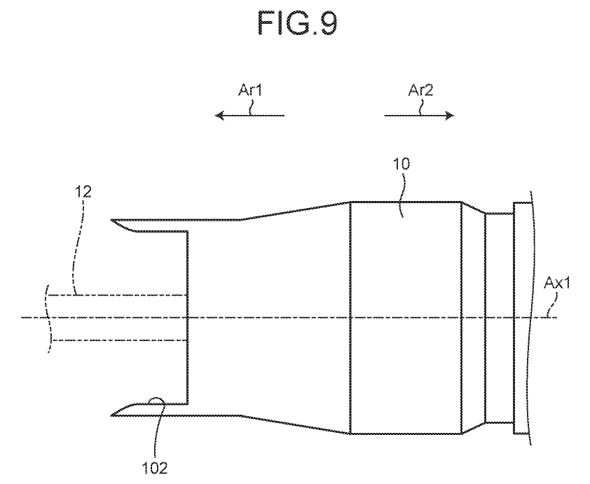

Sixth Modification FIG. 8 and FIG. 9 are diagrams each illustrating a sixth modification of the embodiment. Specifically, FIG. 8 is a perspective view of the end portion of the outer pipe 10 located on the distal end side Ar1 according to the sixth modification. FIG. 9 is a diagram of the end portion of the outer pipe 10 located on the distal end side Ar1 viewed from the side opposite to the side on which the jaw 11 according to the sixth modification is located (the lower side in FIG. 8). Moreover, in FIG. 9, the vibration transmission portion 12 is indicated by an alternate long and short dash line. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. In the sixth modification, as illustrated in FIG. 8 and FIG. 9, in the outer pipe 10, the notch portions 102 and 103 that are described above in the first and the second modifications are provided. Furthermore, in the outer pipe 10, the width size of the notch portion 102 that is provided on the side opposite to the side on which the jaw 11 is located (the lower side in FIG. 8) is larger than the width size of the vibration transmission portion 12, as illustrated in FIG. 9. The width size means the size of the width direction (in the upward and downward directions in FIG. 9). As a result of this, the portion between the end portion of the outer pipe 10 located on the distal end side Ar1 and the treatment portion 121 is electrically insulated in order to prevent a short circuit from occurring by way of the other electrically conductive portion.

Even in a case where the configuration of the above described sixth modification is used, effects similar to those described above in the embodiment are achieved.

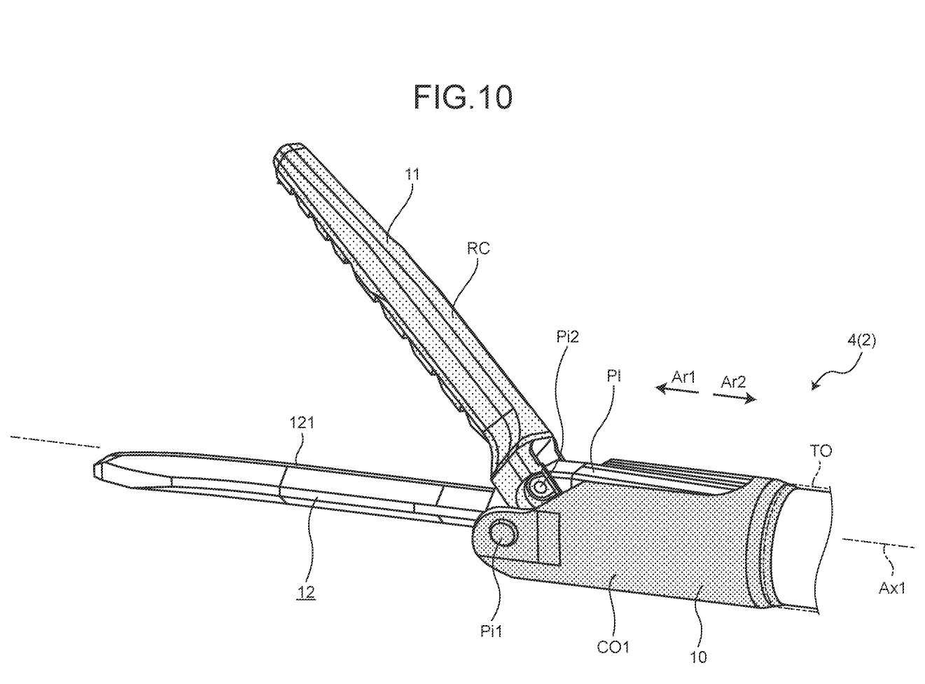

Seventh Modification FIG. 10 is a diagram illustrating a seventh modification of the embodiment. Specifically, FIG. 10 is a diagram corresponding to the diagram illustrated in FIG. 2. In the above described embodiment, as in the seventh modification illustrated in FIG. 10, the side surface of the jaw 11 may be covered by a cover RC. Moreover, in FIG. 10, the cover RC is indicated by dots. The cover RC is made of, for example, polyimide or PEEK that is an electrical insulating material, and is a coating layer that is provided on the surface of the jaw 11 that does not face the treatment portion 121.

Even in a case where the configuration of the above described seventh modification is used, effects similar to those described above in the embodiment are achieved. With the treatment tool 2 according to the seventh modification, the side surface of the jaw 11 is covered by the cover RC. As a result of this, even in a case where another electrically conductive portion is brought into contact over at least one of a portion between the side surface of the jaw 11 and the treatment portion 121 and a portion between the side surface of the jaw 11 and the electrically conductive path to the treatment portion 121, it is possible to prevent a spark from occurring.

Moreover, the cover according to the disclosure is not limited to the coating layer as long as the cover is able to cover the side surface of the jaw 11. For the cover according to the embodiment, it may be possible to use a cover that is formed on the jaw 11 by using an insert molding method, or the like, a cover that is fixed to the jaw 11 by using a snap-fit method, by using a metal pin, or the like.

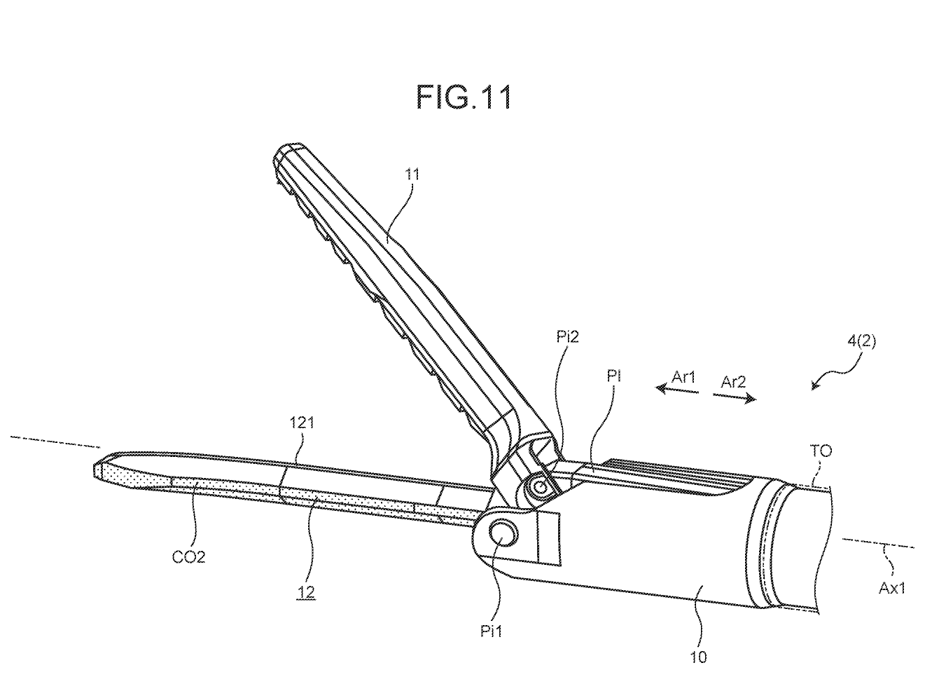

Eighth Modification FIG. 11 is a diagram illustrating an eighth modification of the embodiment. Specifically, FIG. 11 is a diagram corresponding to the diagram illustrated in FIG. 2. In the above described embodiment, the portion between the end portion of the outer pipe 10 located on the distal end side Ar1 and the treatment portion 121 is electrically insulated by the first coating layer CO1 in order to prevent a short circuit from occurring by way of the other electrically conductive portion, but the embodiment is not limited to this. As described in the eighth modification illustrated in FIG. 11, the portion between the end portion of the outer pipe 10 located on the distal end side Ar1 and the treatment portion 121 is electrically insulated by the second coating layer CO2 in order to prevent a short circuit from occurring by way of the other electrically conductive portion.

Specifically, as illustrated in FIG. 11, the second coating layer CO2 may be provided on the outer surface of the treatment portion 121 that does not face the jaw 11. Moreover, in FIG. 11, the second coating layer CO2 is indicated by dots. The second coating layer CO2 is made of, for example, polyimide or PEEK that is an electrical insulating material.

Even in a case where the configuration of the above described eighth modification is used, effects similar to those described above in the embodiment are achieved.

With the treatment tool according to the disclosure, it is possible to prevent a spark from occurring as a result of coming into contact with the other electrically conductive portion.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a vibration transmission portion having a distal end provided with a treatment portion that functions as a first electrode supplying a high frequency current to a biological tissue, the treatment portion being configured to supply ultrasound vibration to the biological tissue;
a jaw configured to be opened and closed with respect to the treatment portion, the jaw functioning as a second electrode supplying the high frequency current to the biological tissue;
a pipe through which the vibration transmission portion is inserted with the treatment portion protruding from a distal end of the pipe, the pipe including an electrically conductive path to the second electrode; and
a support provided on an inner surface of an end portion of the pipe on a distal end side, the support being configured to cover an outer surface of the end portion of the pipe on the distal end side and support the vibration transmission portion,
the support being made of an electrical insulating material such that a portion between the end portion of the pipe on the distal end side and the treatment portion is electrically insulated to prevent a short circuit from occurring by way of another electrically conductive portion.

2. The treatment tool according to claim 1, wherein a first coating layer made of an electrical insulating material is provided on the outer surface of the end portion of the pipe on the distal end side.

3. The treatment tool according to claim 2, wherein
the outer surface of the pipe is covered by a tube made of an electrical insulating material, and
the first coating layer is provided on the outer surface of the end portion of the pipe that is exposed from the tube on the distal end side.

4. The treatment tool according to claim 2, wherein the first coating layer is made of polyimide or polyetheretherketone.

5. The treatment tool according to claim 1, wherein the outer surface of the end portion of the pipe on the distal end side is covered by a tube made of an electrical insulating material.

6. The treatment tool according to claim 5, wherein
a notch portion that is notched from the distal end toward a proximal end of the pipe is provided at the end portion of the pipe on the distal end side, and
the tube covers the outer surface of the end portion of the pipe on the distal end side except for the notch portion.

7. The treatment tool according to claim 1, wherein the pipe is made of an electrical insulating material.

8. The treatment tool according to claim 1, wherein
the pipe includes
a proximal end portion that is made of an electrically conductive material, and that forms the electrically conductive path, and
a distal end portion that is made of an electrical insulating material, and that is provided at an end portion of the proximal end portion on the distal end side.

9. The treatment tool according to claim 1, wherein
the pipe includes
a first pipe, and
a second pipe that is inserted into the first pipe and through which the vibration transmission portion is inserted, and
the second pipe forms the electrically conductive path.

10. The treatment tool according to claim 9, wherein the first pipe is made of the electrical insulating material.

11. The treatment tool according to claim 1, wherein the jaw is pivotally supported with respect to the pipe via an insertion portion made of an electrical insulating material.

12. The treatment tool according to claim 1, wherein
a notch portion that is notched from the distal end toward a proximal end of the pipe is provided at the end portion of the pipe on the distal end side, and
a width size of the notch portion is larger than a width size of the vibration transmission portion.

13. The treatment tool according to claim 1, wherein a side surface of the jaw is covered by a cover made of an electrical insulating material.

14. The treatment tool according to claim 1, wherein a second coating layer made of an electrical insulating material is provided on an outer surface of the treatment portion that does not face the jaw.
